(19) United States
(12) Patent Application Publication
(10) Pub. No.: US 2024/0279293 A1
(43) Pub. Date: Aug. 22, 2024

(54) Method to Produce Soluble Protein Powder Recovered From Organic Waste

(71) Applicants: Figure 8, Inc., San Luis Obispo, CA (US); CIRCULAR UPCYCLING, REDONDO BEACH, CA (US)

(72) Inventor: Ken Tasaki, Redondo Beach, CA (US)

(21) Appl. No.: 18/583,765

(22) Filed: Feb. 21, 2024

Related U.S. Application Data
(60) Provisional application No. 63/576,597, filed on Feb. 21, 2023.

(51) Int. Cl. C07K 14/47; C07K 1/14; C07K 1/34; C07K 1/36

(52) U.S. Cl. CPC C07K 14/4741; C07K 1/145; C07K 1/34; C07K 1/36

(57) ABSTRACT

This invention relates to the field of preparing water-soluble powders of protein extracted from organic waste including, but not limited to manure, food waste, digestate of anaerobic digesters, animal body parts such as hair, wool, nails, skins, feathers, hooves, claws and other body parts by thermal hydrolysis process (THP) without using chemical solvents. The protein includes, but not limited to, keratin, collagen, manure protein, plant proteins. More specifically, the invention relates to process to prepare water-soluble powders of protein recovered from organic waste by adjusting the pH of the extracted solution before undergoing concentration of the solution, removal of water from the solution, finally drying processes such as a spray drying or freeze drying to prepare water-soluble powders.

### CONTINUITY

This application claims priority to and the benefit of co-pending U.S. provisional patent application No. 63/576,597, filed Feb. 21^{st}, 2023, which is incorporated herein by reference in its entirety.

### FIELD OF THE PRESENT INVENTION

This invention relates to the field of preparing water-soluble powders of protein extracted from organic waste including, but not limited to manure, food waste, digestate of anaerobic digesters, animal body parts such as hair, wool, nails, skins, feathers, hooves, claws and other body parts by thermal hydrolysis process (THP) without using chemical solvents. The protein includes, but not limited to, keratin, collagen, manure protein, plant proteins. More specifically, the invention relates to process to prepare water-soluble powders of protein recovered from organic waste by adjusting the pH of the extracted solution before undergoing concentration of the solution, removal of water from the solution, finally drying processes such as a spray drying or freeze drying to prepare water-soluble powders.

### BACKGROUND OF THE PRESENT INVENTION

Keratin and keratin associated protein (KAP) are intracellular proteins in keratinous animal body parts (ABPs) such as hairs, wool, nails, skins, feathers, hooves, claws, and others. The former is hard α-keratins forming microfibrous intermediate filament protein (IFP), while the latter is matrix proteins forming a nonfilamentous matrix. The hard α keratins are highly cross-linked with each other through disulfide bonds, forming a coiled-coil structure with two α-helices making up a protofibril, a bundle of which constitutes a microfibrous IFP which in turn covalently crosslink with KAP. This complex structure makes the hair structure stable and highly resistant to enzymatic degradation. They are a considerable part of slaughtering wastes brought into rendering plants and mostly disposed of, since they are difficult to hydrolyze due to the stable structure described above, thus having poor digestibility as feed.

Given its unique biocompatibility, cell adhesivity, and anti-aging effects on the hairs and skins, the keratin market is growing rapidly. Recently, keratin is increasingly used for hair, skin, and nail care products (Mokrejs, P; Hutta, M.; Pavlackova, J.; Egner, P.; Benicek, L., “The Cosmetic and Dermatological Potential of Keratin Hydrolysate,” J. Cosmet. Dermatol., Feb. 6., 12319 (2017).).

Keratinous ABP (KABP) is an excellent source for keratin, given its high content of keratin on a dry matter basis, >90%.

One key aspect for a keratin product to be commercially successful, whether it is for cosmetics or biomedical applications, is the final form that is a powder.

Powders not only save transportation cost, but a formulator, a manufacturer of the consumer keratin products, can formulate the product, using the keratin at a desirable concentration, be it a haircare or skincare product, according to the formulator's exact proprietary formulation procedure without removing the solvent such as water in which keratin is dissolved by combining with other ingredients.

Furthermore, once the keratin powder is prepared, it can be dissolved in water at a given concentration to prepare a whole host of new compounds through blending or copolymerization with other polymers in aqueous solution, which expands the application of keratin to a wide range of markets (Donato, R. K.; Mija, A. “Keratin Associations with Synthetic, Biosynthetic and Natural Polymers: An Extensive Review,” Polymers 2020, 12, 32; doi:10.3390/polym12010032). Blending or copolymerization are often performed in a liquid phase.

In fact, most keratin ingredient suppliers supply keratin powders to their customers, the keratin manufacturers.

Then, the second key aspect of the keratin products becomes whether or how much the powder dissolves to the solvent the manufacturers use. A 5% solubility in water seems the minimum solubility requirement for keratin powders, according to industrial information.

The problem of the current processes is that they employ highly concentrated, toxic chemicals for the keratin extraction. (Brown, E. M.; Pandya, K.; Taylor, M. M.; Liu, C.-K., “Comparison of Methods for Extraction of Keratin from Waste Wool,” Agricultural Sciences, 7, 670 (2016); Shavandi, A.; Bekhit, A. A.-D.; Carne, A.; Bekhit, A., “Evaluation of Keratin Extraction from Wool by Chemical Methods for Bio-Polymer Application,” J. Bioactive and Compatible Polymers, 1, (2016); Kakkar, P.; Madhan, B.; Shanmugam, G., “Extraction and Characterization of Keratin from Bovine Hoof: A Potential Material for Biomedical Applications,” SpringerPlus, 3, 596 (2014).). These processes are no longer environmentally sustainable.

Most of patent applications or issued patents on extraction of keratin from KABP have also used either chemical methods or enzymatic methods (Toshioka, I.; Kamimura, Y., “Keratin Hydrolyzate Useful as Hair Fixatives,” U.S. Pat. No. 4,390,525A, 1981.; Vermelho, A. B.; Vasquezvilla, A. L.; Mazotto De, A. A. M.; Paraguai De, S. D. E.; Pereira Dos, S. E., “Keratin Hydrolysates, Process for Their Production and Cosmetic Composition Containing the Same,” WO2009000057 A2, 2008; Dupuis, C.; Dubief, C., “Cosmetic Composition for Holding the Hairstyle, Containing a Milk Protein and/or Milk Protein Hydrolysate and a Keratin Hydrolysate,” U.S. Pat. No. 5,679,329A, 1993; Umeda, K.; Nadachi, Y.; Sakai, K.; Nogami, Y.; Sudo, M., “Water-Soluble Keratin Derivative and Use Thereof”US20070128134 A1, 2007; Schrooyen, P.; Boberthur, R., “Keratin-Based Products and Methods for Their Productions,” US20040210039 A1, 2002; Gupta, A.; Ramanan, K.; Kuaman, P., “Process for Extracting Keratin,” US2012/0130048 A1, 2012.). Even enzymatic methods mostly employ chemicals to facilitate the extraction.

According to the recent economic analysis of keratin hydrolysis methods by USDA-ARS, an effective keratin hydrolysis by a combination of chaotropic agent and reducing agent is not promising for economic scale up productions primarily due to the high cost of chemicals (Brown, E. M.; Pandya, K.; Taylor, M. M.; Liu, C.-K., “Comparison of Methods for Extraction of Keratin from Waste Wool,” Agricultural Sciences, 7, 670 (2016).).

Alternative processes such as microwave extraction, steam explosion process, and ionic liquid process. (Ji, Y.; Chen, J.; Lv, J.; Li, Z.; Xng, L.; Ding, S., “Extraction of Keratin with Ionic Liquids from Poultry Feather,” Separation and Purification Technology, 132, 577 (2014).; Marina Zoccola, Annalisa Aluigi, Alessia Patrucco, Claudia Vineis, Fabrizio Forlini, Paolo Locatelli, Maria Carmela Sacchi and Claudio Tonin, “Microwave-Assisted Chemical-Free Hydrolysis of Wool Keratin,” Bioresource Technology, 70, 111 (1999)). They all have problems such as the high recovery cost, the scaling up issues, and low recovery yields. No report of making powders using these alternative methods has been published.

THP uses only water as the extraction solvent; hence, minimizing the environmental risk, as is the case for chemical extraction processes. Since it does not use chemicals, THP does not require complex recovery processes for recycling of chemicals, potentially reducing the production cost and offering a simple, alternative, environmentally sustainable process.

THP has been applied to extract keratin from feather, hog hair, and wool. [Yin, J.; Rastogi, S.; Terry, A. E.; Popescu, C., “Self-Organization of Oligopeptides Obtained on Dissolution of Feather Keratins in Superheated Water,” Biomacromolecules, 8, 800 (2007).; Esteban, M. B.; Garcia, A. J.; P. Ramos; Marquez, M. C., “Sub-Critical Water Hydrolysis of Hog Hair for Amino Acid Production,” Bioresource Technology, 101, 2471 (2010).; Bhavsar, P.; Zoccola, M.; Patrucco, A.; Montarsolo, A.; Rovero, G.; Tonin, C., “Comparative Study on the Effects of Superheated Water and High Temperature Alkaline Hydrolysis on Wool Keratin,” Textile Res. J, Jul. 7, 2016.]

None of the previously reported THP studies describe the preparation of powders except for the article by Tasaki. [K. Tasaki “A novel thermal hydrolysis process for extraction of keratin from hog hair for commercial applications,” Waste Management 104 (2020) 33-41]

We examined the solubility of the powder using the method described in the article by Tasaki. We show in this application that the powder extracted by THP and simply dried by a dryer after concentrating the extracted keratin solution has little solubility in water.

High concentration solutions of keratin are often required for testing or evaluation.

For example, wound-care efficacy tests often require more than 5% of keratin concentration. Likewise, haircare efficacy tests also require about the same level of concentration.

For commercial applications, keratin manufacturers often prefer powders, as is mentioned above.

However, we have often experienced a difficulty to increase the keratin concentration higher than 2 wt % by THP extraction of keratin.

We have invented a method not only to increase the keratin concentration, but to prepare a water-soluble keratin powder after the keratin is extracted by THP from KABP and the extracted keratin solution is concentrated by a simple procedure. In particular, the method employs adjustment of pH of the extracted keratin solution.

After extensive literature search, we found no reports on the controlling of solubility of the extracted keratin by adjusting pH of the extracted keratin solution. Most patents on keratins focus on either the extraction or the application, not controlling the solubility of the extracted keratin.

For example, U.S. Pat. No. 7,148,327 B2 is an important patent on extraction of keratin from KABP based on which several popular keratin products have been produced. No mention of the water solubility of the extracted keratin is made, and no use of pH to control the solubility is described (Kelly, R. J. et al. “Production of Soluble Keratin Derivatives,” U.S. Pat. No. 7,148,327 B2, Dec. 12, 2006.).

Even for the preparation of keratin film after the keratin extraction, pH was considered for the precipitation of the film, rather than the solubility increase of extracted keratin in water to form a film (Tanabe, T.; Okitsu, N.; Tachibana, A.; Yamauchi, K. “Preparation and characterization of keratin-chitosan composite film,” Biomaterials (2002) 23, 817-825).

Most literatures including patents discuss the keratin solubility in the context of keratin extraction, rather than manipulating the solubility of the extracted keratin which is also important once keratin extracted (Perţa-Crişan, S.; Ursachi, C. Ş.; Gavrilaş, S.; Oancea, F.; Munteanu, F.-D. “Closing the Loop with Keratin-Rich Fibrous Materials,” Polymers (2021) 13, 1896. https://doi.org/10.3390/polym13111896).

We found one article in which the stability of an extracted keratin protein solubility in water was modified through chemical functionalization, more complex than a simple pH adjustment (Saleknezhad, M.; Robatjazi, S.; Morteza, S.; Zeinoddini, M. (2021). “Increasing the stability of keratin protein solubility in aqueous solutions using the chemical structure modification by alkylation and sulfitolysis methods. New Cell. Mol. Biotech. (2021) 11 (41), 29-45. SID. https://sid.ir/paper/977056/en).kali or acid extraction of

THP can be applied to recover proteins from other organic waste. For example, we found one study using THP to release protein from manure; however, it still uses chemicals as a catalyst. [Xie, S., Zhang, T., Mishra, A., Tiwari, A., & Bolan, N. S. “Assessment of catalytic thermal hydrolysis of swine manure slurry as liquid fertilizer: Insights into nutrients and metals,” Frontiers in Environmental Science, 10, 1005290 (2022). No recovery or isolation of protein was described in the study.

After an extensive literature search, we failed to find any publication to describe both extraction and isolation of protein from livestock manure without using any chemicals in the literature.

Another example would be protein recovery from sludges at wastewater treatment facilities (WWTF). The industry has a growing demand for recovering protein from protein-rich sludges.

One study reports a recovery of protein from sludges produced at a WWTFs by THP. [García, M., Urrea, J. L., Collado, S., Oulego, P., & Díaz, M. “Protein recovery from solubilized sludge by hydrothermal treatments,” Waste Management, 67, 278-287 (2017). A precipitation method by ammonium sulfate was chosen for the separation of protein from the hydrolyzed sludge. However, such precipitation generally requires a large amount of ammonium sulfate. No mention of the protein solubility is made in the article.

### SUMMARY OF THE PRESENT INVENTION

The present invention describes a method in which soluble powders of keratin extracted from KABP can be prepared by adjustment of pH in the extracted keratin solution. This adjustment of pH in the extracted keratin solution is referred to as “the pH adjustment.”

### BRIEF DESCRIPTION OF DRAWINGS

FIG. 1-FIG. 1 illustrates the aqueous solutions after mixing the keratin powders in water without the pH adjustment: (a) the solution mixed with the keratin powder prepared at 160° C.; (b) the solution mixed with the keratin powder prepared at 180° C.; (c) the solution mixed with the keratin powder prepared at 200° C.

FIG. 2-FIG. 2 illustrates the solid precipitate left at the bottom of the flask after the solution with the powder prepared at 200° C. is removed when the powder was prepared without the pH adjustment.

FIG. 3 (a)-FIG. 3 shows the solution after mixing the powder prepared at 160° C. with the pH adjustment: (I) the flask bottom from outside; (II) the flask bottom looking inside.

FIG. 3 (b)-FIG. 3 shows the solution after mixing the powder prepared at 180° C. with the pH adjustment: (I) the flask bottom from outside; (II) the flask bottom looking inside.

FIG. 3 (c)-FIG. 3 shows the solution after mixing the powder prepared at 200° C. with the pH adjustment: (I) the flask bottom from outside; (II) the flask bottom looking inside.

FIG. 4-FIG. 4 shows the solution after mixing the powder prepared at 200° C. with the pH adjustment and then stored at 2° C. in a refrigerator: (I) the flask bottom from outside; (II) the flask bottom looking inside.

FIG. 5-FIG. 5 displays the concentrated keratin solution (a) with and (b) without the pH adjustment.

FIG. 6-FIG. 6 illustrates the inhibition (%) of the damage by the hydroxyl radical by (a) the keratin powder and (b) Trolox as a function of the logarithm of the concentration in mg/L.

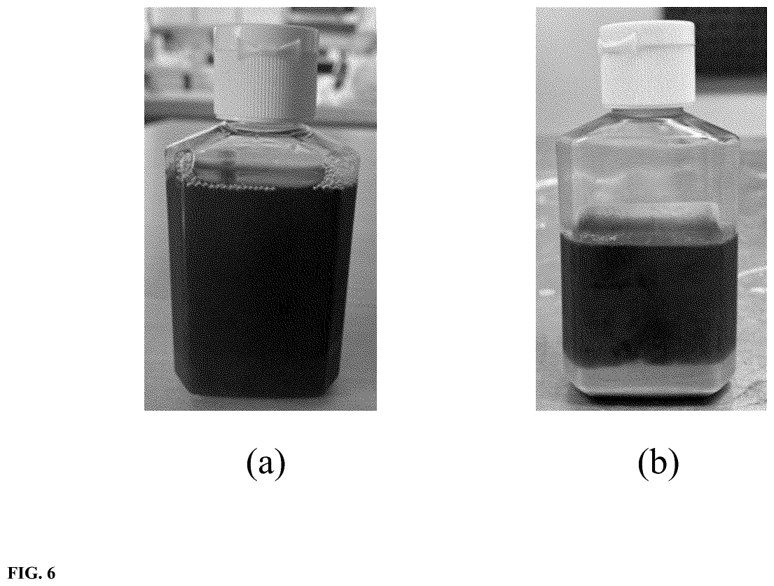

### DESCRIPTION OF THE PREFERRED EMBODIMENT(S) OF THE PRESENT INVENTION

The detailed description set forth below is intended as a description of presently-preferred embodiments of the invention and is not intended to represent the only forms in which the present invention may be constructed and/or utilized. However, it is to be understood that the same or equivalent functions and results may be accomplished by different embodiments that are also intended to be encompassed within the spirit and scope of the invention, and additional variations of the present invention may be devised without departing from the inventive concept.

### DETAILED DESCRIPTION OF INVENTION

The present invention discloses a method in which a soluble powder of keratin or keratin hydrolysate can be prepared by adjusting pH of the keratin extraction solution before drying the solution by spray drying or freeze drying, but after keratin is extracted from KABP and hydrolyzed by THP and recovered by ultrafiltration (UF), or more preferably using anti-membrane fouling ultrafiltration (AFUF) which includes, but not limited to, the shear wave-induced ultrafiltration or the vibratory shear-enhanced process.

When the content of organic matters in waste is high, they tend to cause membrane fouling which requires frequent washing and replacing membranes which add operating cost. It is preferable to use AFUF when the content of organic matters is high. (→ the old Claim 4, the new Claim 5.) UF is to remove suspended solids (SS) in the effluent from THP. The size of SS is generally >1 mm which is about 5,000 KDa in the molecular weight cutoff (MWCO).

On the other hand, the molecular weight (MW) distribution of the dissolved keratin was found to be up to around 100 KDa in our bench-scale experiments. To make sure to have no SS in the UF permeate and recover all extracted keratin, the MWCO of 150 KDa may be preferred for the membrane pore size. In the bench-scale experiments, it was also found that there were some fractions of extracted keratin below 5,000 Da. These MW fraction can be recovered by NF with MWCO of less than 6,000 Da.

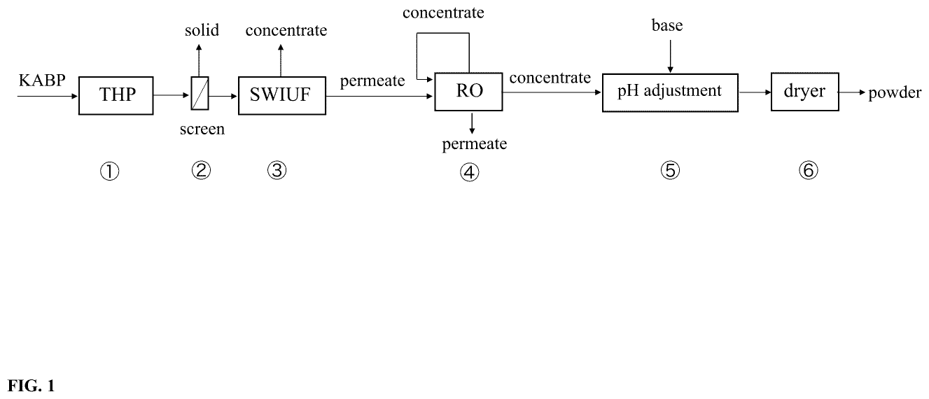

More specifically, the invented process consists of the following steps: \textcircled{1} Keratin is extracted from KABP by THP; \textcircled{2} the THP effluent is filtered by a screen with a 20˜100 mm mesh; \textcircled{3} the filtrate is further filtered by UF or AFUF; \textcircled{4} the UF or the AFUF permeate is concentrated by RO in a way that the concentrate from RO is circulated back to RO, while water is removed through the permeate, until the permeate ceases to stop; \textcircled{5} the pH of the final concentrate from RO is adjusted to be higher than or equal to 8, but lower than 10 by adding base; \textcircled{6} the solution is dried by a dryer to make a powder. See FIG. 1 for the process flow diagram.

The solution of keratin extracted by THP from KABP has pH of less than 6 in general.

In THP treatments, KABP is heated in water at high temperatures under which water acts more as a hydrophobic solvent interfering with forces acting on IFP to loosen the fibril network, which is tightly bundled through hydrophobic interactions. This behavior of water has a swelling effect on the IFP network.

Once swollen, pores are created in the IFP network. When the pores become large enough, the hydronium ions dissociated from water at high temperatures can penetrate the pores through which the hydronium ions reach the disulfide bonds for the bond cleavage, releasing individual keratin protein molecules into water.

Once dissolved in water, the protein may undergo reconfiguration of the three-dimensional structure in such a way that the hydrophobic groups are exposed to water which acts more as a hydrophobic solvent. The keratin extraction by THP can be performed by controlling either the temperature (Tᵢ) or the pressures (Pᵢ) or both for a certain time (tᵢ) for a given number of steps (i=1˜5). tᵢ is referred to as the reaction time. For example, when i=2, Tᵢ ranges from 100° C. to 200° C. when Tᵢ<T₂. Pᵢ may range from 10 to 30 MPa when Pᵢ<P₂.

When Tᵢ is above 200° C., the amino acid residues in the keratin start reacting one another. Hence, Tᵢ should be ≤200° C. On the other hand, 100° C. may be often too low to break the disulfide bonds sufficiently. Thus, a preferred Tᵢ should be between 160° C. and 200° C.

The reaction time should be long enough to complete the dissolution of keratin from KAB, but not too long to avoid the internal reaction among the amino acid residues. A preferred time may be 1 to 2 hrs.

Even after the water temperature decreases to ambient temperature, the same protein structure may stay, having little solubility in water, with the hydrophobic groups on the surface, wrapping around the hydrophilic groups inside.

It is known that the protein structure is sensitive to pH, transforming its structure. By adjusting pH of the keratin solution, the keratin protein can rearrange itself in such away that the hydrophilic groups extend themselves to water, while wrapping the hydrophobic groups inside.

In fact, pH of solutions of keratin extracted by THP tends to have a pH lower than 6. When the pH of the keratin solution is increased to 7 or 8 by adding a base, the keratin powder dried from the solution becomes water soluble. However, if pH increases further above 10, the protein may undergo hydrolysis. Hence, the pH of the solution should not increase above 10.

It is also known that a protein has a minimum solubility in water at the pH corresponding to its isoelectric point (pl). It has been reported that the pls of alpha keratin ranged from 4.5 to 7.0 (Toni, M.; Alibardi, L. “Alpha- and beta-keratins of the snake epidermis,” Zoology (Jena, Germany) (2007); 110(1):41-47. DOI: 10.1016/j.zool.2006.07.001.). For these keratins, a base should be used to increase the value of pH before making powders.

On the other hand, the pls of beta keratin have the values of 6.5˜8.5 (Alibardi L, Toni M. Characterization of keratins and associated proteins involved in the corneification of crocodilian epidermis. Tissue Cell. 2007 October; 39(5):311-23.). For these keratins, an acid should be used to increase the value of pH before making powders.

The pH of the extracted keratin solution prepared by this invention ranged from 5.4 to 5.7 before adjusting pH. These numbers are within the pl published for alpha keratin which includes human hair.

This invention is not the first to adjust the water solubility of keratin. A patent U.S. Pat. No. 2,993,794 used a pH adjustment to control the solubility of keratin (Moshy, R. J. “Process for Preparing Keratin Protein,” U.S. Pat. No. 2,993,794, 1961.). However, the adjustment of pH was rather to precipitate the keratin, extracted using dialkyl sulfoxides as the extraction solvent, through bringing the pH close to the pl of keratin for recovery, than dissolving the keratin.

In addition, controlling pH is often used to extract individual keratin protein molecules from KABP, mostly through increasing pH by using alkali, referred to as the Alkali Method, but not to increase the solubility of individual keratin proteins in water after the extraction. Wang et al. have used alkali to increase pH of the extraction solution to dissolve keratin molecules from KABP (Wang, X.; Shi, Z.; Zhao, Q.; Yun, Y. “Study on the Structure and Properties of Biofunctional Keratin from Rabbit Hair,” Materials 2021, 14, 379. https://doi.org/10.3390/ma 14020379), as is the case for other studies (Horvath: Solubility of Structurally Complicated Materials: 3. Hair The Scientific World JOURNAL (2009) 9, 255-271).

When pH shifts away from the protein's pl, the solubility of the protein increases. However, if pH is too high, e.g., >10, or too low, e.g., <4, the protein tends to undergo hydrolysis which should be avoided to maintain the molecular weight (MW) of the original protein. Hence, when the pl of the protein is low, increasing pH up to 8, but below 10, may be preferable. Likewise, when the pl of the protein is high, lowering pH down to 5, but above 4, may be preferable.

After extensive search, we have failed to find any literature regarding the pH adjustment to increase the solubility of extracted keratin.

In one embodiment, keratin is extracted by THP from KABP and the extracted keratin solution is filtered by screen with a 20˜100 mm mesh to remove the unreacted solid. The filtrate is further filtered by UF or AFUF to remove suspended solids (SS). The permeate from UF or AFUF is filtered by RO to remove water by circulating the concentrate through RO repeatedly until the permeate from RO creases to stop. Then, the pH of the RO concentrate solution is adjusted to be higher than or equal to 8, but lower than 10 by adding a base before a drying process to make a powder.

More generally, in another embodiment, keratin is extracted by THP from KABPs and the extracted keratin solution is filtered by screen with a 20˜100 mm mesh to remove the unreacted solid. The filtrate is further filtered by UF or AFUF to remove suspended solids. The permeate from UF or AFUF is filtered by RO to remove water by circulating the concentrate through RO repeatedly until the permeate from RO creases to stop. Then, the pH of the RO concentrate solution is adjusted so that the pH shifts away from the keratin's pl by adding either acid or base before a drying process to make a powder. For example, if the keratin's pl is 5, the pH should be increased to around or higher 8 by adding base. Conversely, if the keratin's pl is 8, the pH should be decreased to around 5 or lower by adding acid.

In another embodiment, keratin is extracted by THP from KABP and the extracted keratin solution is filtered by screen with 20˜100 mm mesh to remove the unreacted solid. The filtrate is further filtered by UF or AFUF to remove suspended solids. The pH of the UF or the AFUF permeate is adjusted to be higher than or equal to 8, but not above 10 by adding a base before concentrating the solution by using nanofiltration (NF) in which the NF concentrate is circulated through NF repeatedly until the permeate from NF ceases to stop. No precipitation of solid occurs after the pH adjustment. The concentration can be increased by adjusting the pore size of NF membrane.

More generally, in another embodiment, keratin is extracted by THP from KABP and the extracted keratin solution is filtered by screen with 20˜100 mm mesh to remove the unreacted solid. The filtrate is further filtered by UF or AFUF to remove suspended solids. The pH of the UF or the AFUF permeate is adjusted so that the pH shifts away from the keratin's pl by adding either acid or base before concentrating the solution by using nanofiltration (NF) in which the NF concentrate is circulated through NF repeatedly until the permeate from NF ceases to stop. No precipitation of solid occurs after the pH adjustment. The concentration can be increased by adjusting the pore size of NF membrane. For example, if the keratin's pl is 5, the pH should be increased to around 8 by adding base. Conversely, if the keratin's pl is 8, the pH should be decreased to around 5 by adding acid.

Yet in another embodiment, the protocol we invented, the pH adjustment, should be able to apply to the keratin extracted by any methods including chemical methods, the enzymatic methods, the other alternative methods described in this application. For example, after keratin is extracted by chemical methods including, but not limited to, the sulfitolysis, the reduction method, the alkaline hydrolysis, and the oxidation, and the extracted keratin solution is purified by purification including, but not limited to, dialysis, UF, and centrifugation, then, the pH of the purified keratin solution is adjusted in a way that the pH shifts away from the keratin's pl by adding either acid or base, before the solution is concentrated and made into a powder through drying process including lyophilization, spray drying, and freeze drying. For example, if the keratin's pl is 5, the pH should be increased to around 8 by adding base. Conversely, if the keratin's pl is 8, the pH should be decreased to around 5 by adding acid.

The same method can be applied to recovery of other proteins and produce a water-soluble protein. For example, ABP contains a large volume of collagen which has a variety of commercial applications including cosmetics and biomedical applications.

Likewise, a U.S. patent, U.S. Ser. No. 10/150,711 B2, claims an extraction of protein from manure. [Vanotti et al. “Extraction of Amino Acids and Phosphorus from Biological Materials,” U.S. patent Ser. No. 10/150,711 B2, Dec. 11, 2018.]. It makes heavy use of alkali as the extraction solvent. Also, neither isolation nor solubility of protein is described in the patent.

Extraction of protein from manure may have two benefits: 1) the protein in manure is otherwise known as the organic nitrogen which is very difficult to breakdown by conventional biological treatment processes, hence staying in the soil for a long period of time which results in runoff to rivers, lakes, and bays, causing eutrophication.

The other benefit may be to use the extracted protein as feed additives or biostimulants whose markets are growing rapidly.

The protein recovered by THP is hydrolyzed during the THP treatment; hence, they are protein hydrolysates (PHs). Many PHs have been found to have antioxidants. [Adhikari, B.; Dhungana, S. K.; Ali, M. W.; Adhikari, A.; Kim, I.-D.; Shin, D.-H., “Antioxidant Activities, Polyphenol, Flavonoid, and Amino Acid Contents in Peanut Shell,” J. the Saudi Soc. Agricultural Sciences (2018); https://doi.org/10.1016/j.jssas.2018.02.004; Liu, R.; Xing, L.; Fu, Q.; Zhou, G.-H.; Zhang, W.-G., “A Review of Antioxidant Peptides Derived from Meat Muscle and By-Products,” Antioxidants, 5, 32 (2016); Feng, P.; Ding, H.; Lin, H.; Chen, W., “AOD: the Antioxidant Protein Database,” Scientific Reports, 7, 7449 (2017), DOI:10.1038/s41598-017-08115-6; Ye, N.; Hu, P.; Xu, S.; Chen, M.; Wang, S.; Hong, J.; Chen, T.; Cai, T., “Preparation and Characterization of Antioxidant Peptides from Carrot Seed Protein,” J. Food Quality, 2018, Article ID 8579094, https://doi.org/10.1155/2018/8579094; Kim, J. M.; Liceaga, A. M.; Yoon, K. Y., “Purification and Identification of an Antioxidant Peptide from Perilla Seed (Perilla frutescens) Meal Protein Hydrolysate,” Food Sci. Nutr. 1 (2019).] Accordingly, the protein recovered by our THP method can be used as antioxidants.

Many PHs have also been found to be effective as biostimulants. [Fedoreyeva, L. I. “Molecular Mechanisms of Regulation of Root Development by Plant Peptides,” Plants, 12(6), 1320 (2023). https://doi.org/10.3390/plants12061320; Hu, Z., Zhang, H., & Shi, K. “Plant peptides in plant defense responses,” Plant Signaling & Behavior, 13(8) (2018). https://doi.org/10.1080/15592324.2018.1475175; Trovato, M., Funck, D., Forlani, G., Okumoto, S., & Amir, R. “Editorial: Amino Acids in Plants: Regulation and Functions in Development and Stress Defense,” Frontiers in Plant Science, 12, 772810 (2021). https://doi.org/10.3389/fpls.2021.772810; Yang, Q., Zhao, D., & Liu, Q. “Connections Between Amino Acid Metabolisms in Plants: Lysine as an Example,” Frontiers in Plant Science, 11, 548590 (2020). https://doi.org/10.3389/fpls.2020.00928.] Hence, the protein recovered by our THP method can be used as biostimulants.

The protein hydrolysates are increasingly in high demands as feed additives. [ ] the protein recovered by our THP method can be used as feed additive.

Rich sources of PH include manure, microalgae, KABP, and ABP.

### Example 1

20 g of human hair was washed in 5% detergent in 1 L of water through agitation. The rinsed human hair mixed in 1 L of deionized water was placed in a THP reactor vessel and heated at 160, 180, and 200° C. for 1 hr separately. The pressure was the corresponding saturated vapor pressure at each temperature. The effluent from the THP reactor vessel was first filtered by a 20 mm-mesh screen, followed by AFUF to remove suspended solid. The permeate from AFUF was concentrated by circulating the concentrate from RO, hence removing water from the AFUF permeate. The circulation was repeated until no permeate comes out from RO. The concentrated solution by RO was dried by a spray dryer to make a powder without adjusting the pH. pHs of these solutions ranged from 5.4 to 5.7. The solubilities of the powders prepared at different THP temperatures were examined by mixing a given mass of the powder with 200 mL of water by a stirrer for 10 min. and then inspecting the color of the solution and precipitations at the bottom of the flask.

Table 1 lists the solubilities of the powders prepared as described above.

TABLE 1
Solubilities of Keratin Powders prepared under Different
THP Temperatures without pH Adjustment.

| | 160° C. | 180° C. | 200° C. |
|---|---|---|---|
| Solubility, g/L | 0.10 ± 0.01 | 0.08 ± 0.01 | 0.05 ± 0.01 |

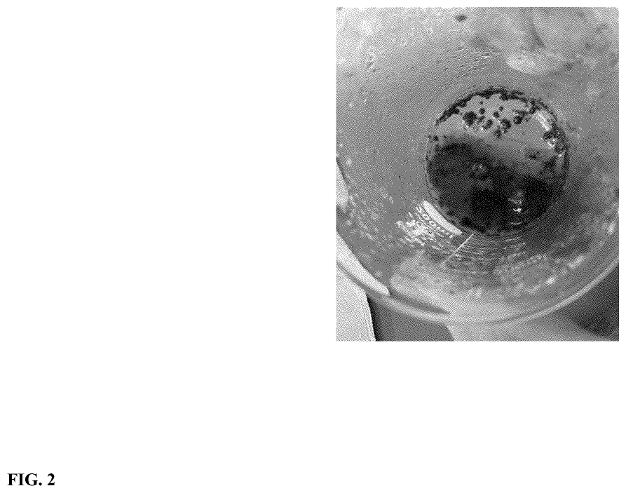

As Table 1 indicates, very little amount of the powders were dissolved in water. The solubility appeared to increase as the temperature decreased. FIG. 1 illustrates the solutions after mixing each powder in water. Precipitates are visible at the bottom of each flask. The colors of the solutions are light, given the low solubilities. FIG. 2 shows the precipitate at the bottom of the flask after removing water for the solution which was prepared by mixing the powder produced by THP at 200° C.

### Example 2

The concentrated keratin solutions were prepared as were described in EXAMPLE 1 under the same conditions. Before drying the solutions, pH of each concentrated solution by RO was adjusted to be 7 by adding 1 M NaOH.

The solubility of each powder was examined as is described above. Table 2 lists the solubilities of keratin powders prepared as described above.

TABLE 2
Solubilities of Keratin Powders prepared under
Different THP Temperatures with pH Adjustment.

| | 160° C. | 180° C. | 200° C. |
|---|---|---|---|
| Solubility, g/L | 1.02 ± 0.01 | 0.91 ± 0.01 | 0.35 ± 0.01 |

The solubilities of the powders increased by an order of magnitude, compared to those in EXAMPLE 1. The effect of the pH adjustment is clear. However, the keratin concentration may not be high enough for some keratin manufacturers.

### Example 3

The concentrated keratin solutions were prepared as were described in EXAMPLE 1 under the same conditions. pHs of these solutions ranged from 5.4 to 5.7. Before drying the solutions, pH of each concentrated solution by RO was adjusted to be 8 by adding 1 M NaOH.

The solubility of each powder was examined as is described above. Table 2 lists the solubilities of keratin powders prepared as described above.

TABLE 2
Solubilities of Keratin Powders prepared under
Different THP Temperatures with pH Adjustment.

| | 160° C. | 180° C. | 200° C. |
|---|---|---|---|
| Solubility, g/L | 11.62 ± 0.01 | 11.41 ± 0.01 | 11.39 ± 0.01 |

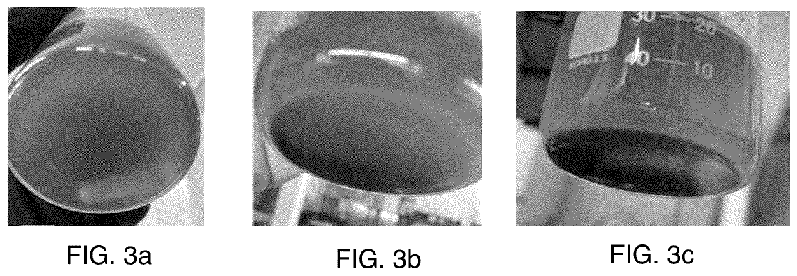

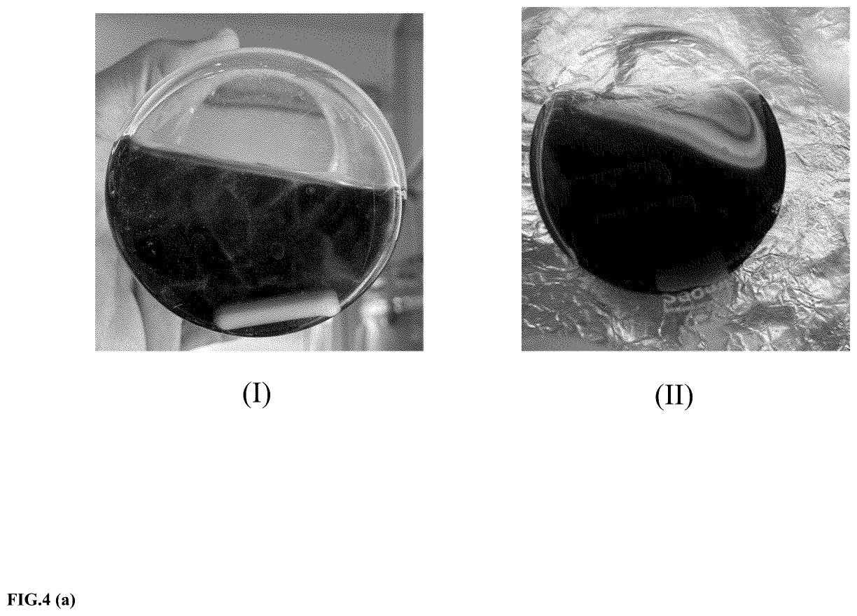

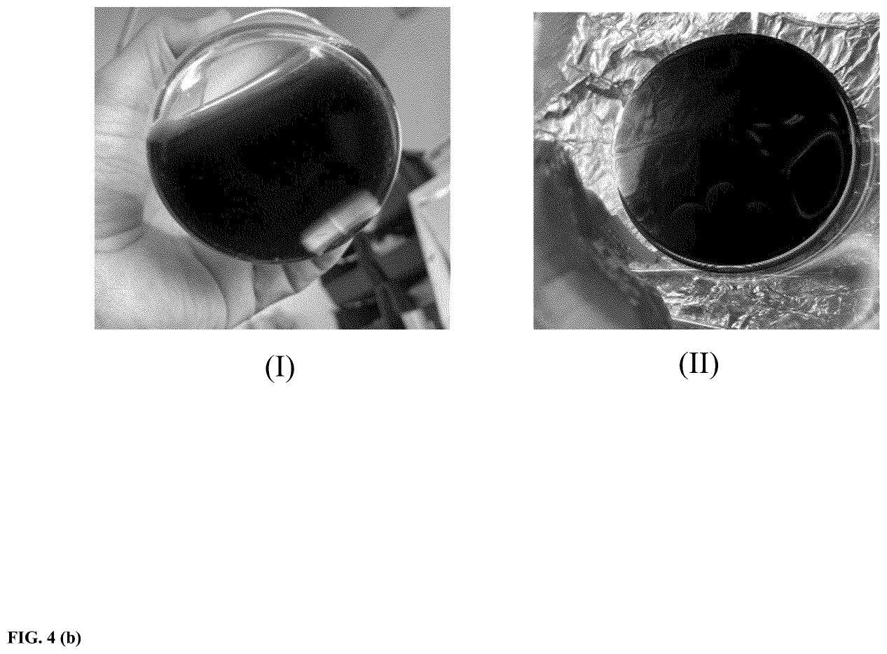

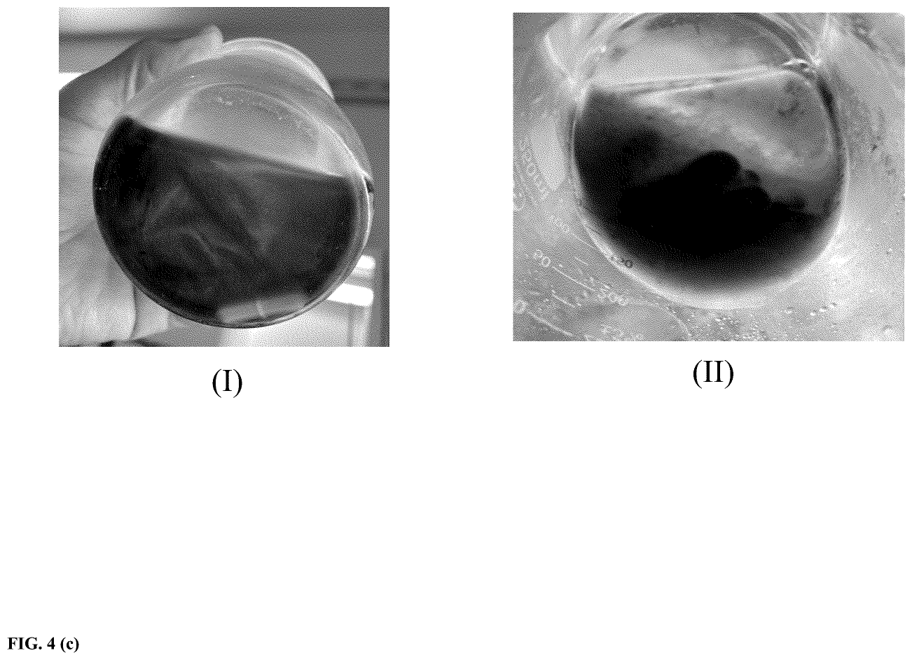

The solubilities of the powders significantly improved by two orders of magnitude, compared to those in EXAMPLE 1. The effect of the pH adjustment is clear. FIG. 3 (a)˜(c) illustrates the solutions after mixing the powders prepared by the pH adjustment described above. No precipitate was observed at the bottom of the flask for each solution. The color of the solutions are darker than those in FIG. 1 because the keratin concentrations were higher than those in FIG. 1. FIG. 4 shows the solution after storing at 2° C. in a refrigerator overnight. No sign of precipitation is observed.

### Example 4

20 g of feather in 1 L of deionized water was placed in a THP reactor vessel and heated at 160° C. for 1 hr. The pressure was the corresponding saturated vapor pressure at each temperature. The effluent from the THP reactor vessel was first filtered by a 20 mm-mesh screen, followed by AFUF to remove suspended solid. The pH of the AFUF permeate is adjusted to be 8 by adding a base before concentrating the solution by using nanofiltration (NF) in which the NF concentrate is circulated through NF repeatedly until the permeate from NF ceases to stop. The concentration can be increased by adjusting the pore size of NF membrane. This sample is referred to as Sample I.

Another NF concentrate keratin solution was prepared as is described above without any pH adjustment. This sample is referred to as Sample II.

Table 4 lists the solubility of Sample I and II.

TABLE 4
Solubility of Sample I and II

| | Sample I | Sample II |
|---|---|---|
| Solubility, g/L | 10.36 ± 0.01 | 3.42 ± 0.01 |

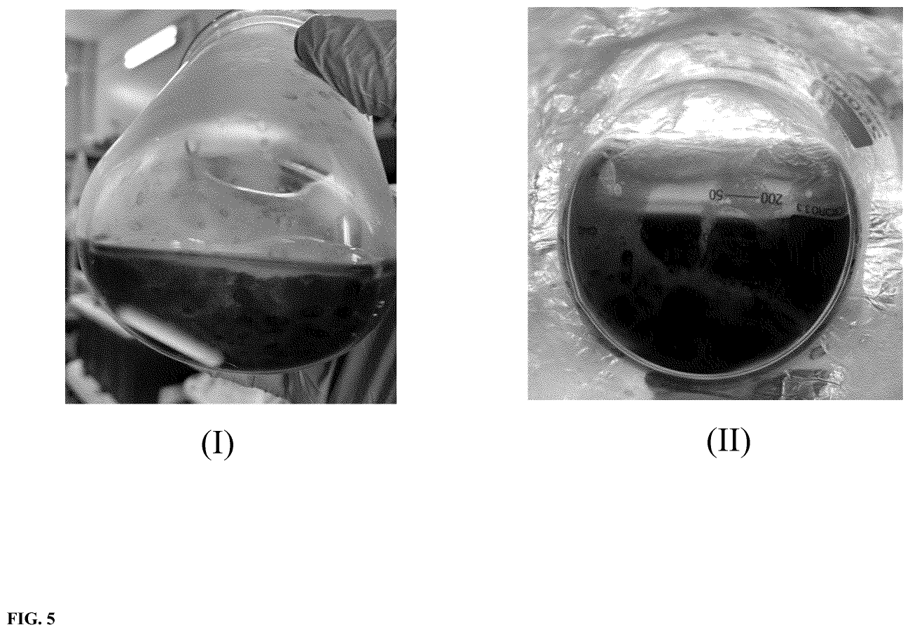

The solubility of Sample I with the pH adjustment has three-times as high as that of Sample II. FIG. 5 shows the solution (a) with and (b) without the pH adjustment. Solution (a) has no visible precipitation, while Solution (b) has a clear precipitation at the bottom of the vial.

### Example 5

The antioxidant activity of the keratin powder was evaluated by the oxygen radical absorption capacity assay by measuring the fluorescence of a protein called fluorescein in the presence of a given concentration of the hydroxyl radical and the keratin powder. The stronger the antioxidant activity of the subject is, the higher the fluorescence signal becomes, exhibiting a higher inhibition of the damage to the protein by the radicals. FIG. 7 (a) shows the inhibition of hydroxyl radicals in percentage as a function of the keratin powder in the logarithm. A 100% inhibition was achieved when the keratin powder logarithm concentration was around 2.5. FIG. 7 (b) displays the inhibition by Trolox, an analogue of vitamin E for comparison. It reached a 100% inhibition when the logarithmic concentration of Trolox was 3.3. The lower concentration of the keratin powder was required to reach 100% inhibition than Trolox. Further, IC₅₀, the concentration that reduces the inhibition by a half, of the keratin powder was 107 mg/L, while IC₅₀ of Trolox was 741 mg/L. These observations suggest that the keratin powder has a higher antioxidant activity than Trolox, as much as seven times.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

I claim:

1. A method of producing soluble protein powder from organic waste, comprising the steps of:
(1) extracting protein by thermal hydrolysis process (THP) to create THP effluent from organic waste by controlling the temperature (Tᵢ), the pressure (Pᵢ), and the reaction time (tᵢ) over several steps where i=1˜5;
(2) filtering of the THP effluent to form filtrate using a mesh screen;
(3) ultrafiltering the filtrate to produce ultrafiltered permate;
(4) removing water from the ultrafiltered permeate by reverse osmosis to obtain reverse osmosis concentrate;
(5) adjusting the pH of the reverse osmosis concentrate to render reverse osmosis concentrate into a final powder that is water soluble;

2. The method of claim 1, wherein the pH of the said concentrated solution is adjusted so that the pH shifts away from the protein's isoelectric point by adding either base or acid before drying the solution.

3. The method of claim 1, wherein the method produces a concentrated protein solution by nanofiltration or reverse osmosis or combination thereof, without making powders.

4. The method of claim 1, wherein the reverse osmosis concentrate is dried by a drying process to make water-soluble protein powders.

5. The method of claim 1, wherein the ultrafilter is AFUF.

6. The method of claim 1, wherein the pore size employed for said ultrafilter is 150 KDa.

7. The method of claim 1, wherein the pH of the reverse osmosis concentrate is adjusted to be higher than or equal to 8, but lower than 10 by adding an alkali to the solution, before drying the solution by a drying process for powder preparation.

8. The method of claim 1, wherein the molecular weight fraction of the said keratin below 5,000 Da is recovered by nanofiltration through adjusting the membrane pore size.

9. The method of claim 1, wherein the THP is performed at the temperature equal to or higher than 160° C., but lower than or equal to 200° C. for 2 hrs.

10. The method of claim 1, wherein the THP is performed at the temperature equal to or higher than 160° C., but lower than or equal to 200° C. for 1 hr.

11. The method of claim 1, wherein the protein that is extracted from organic waste is keratin; and wherein the organic waste is ABP.

12. The method of claim 1, wherein the protein that is extracted from organic waste is collagen; and wherein the organic waste is ABP.

13. The method of claim 1, wherein the organic waste is animal manure.

14. The method of claim 1, wherein the organic waste is microalgae.

15. The method of claim 1, wherein the protein powder is antioxidant.

16. The method of claim 1, wherein the protein powder is biostimulant.

17. The method of claim 1, wherein the protein powder is feed additive.

18. The method of claim 7, wherein the protein powder is a keratin powder for a wound dressing ingredient.

19. The method of claim 7, wherein the said protein powder is a keratin powder for a cosmetics ingredient.

20. The method whereby protein is extracted, comprising:
extracting the protein by a chemical process, the chemical process selected from the following group: sulfitolysis, alkali hydrolysis, oxidation, and reduction;
purifying the extracted protein by a purification process to form a purified extracted protein, the purification process selected from the following group: dialysis, UF, and centrifugation;
adjusting the pH of the purified extracted protein so that the pH shifts away from the protein's isoelectric point by adding either base or acid; and
converting the purified extracted protein to a powder by a drying method selected from the following group: lypholization, spray drying, and a freeze drying.
